(19) United States
(12) Patent Application Publication
(10) Pub. No.: US 2013/0035375 A1
(43) Pub. Date: Feb. 7, 2013

(54) Gene re-cycling

(71) Applicant: Zachariah Mathew, West Orange, NJ (US)

(72) Inventor: Zachariah Mathew, West Orange, NJ (US)

(73) Assignee: Dr. Mrs. Thankam Mathew, M.V.Sc., Ph.D., West Orange, NJ (US)

(21) Appl. No.: 12/586,869

(22) Filed: Sep. 29, 2009

Related U.S. Application Data
(60) Provisional application No. 61/194,637, filed on Sep. 30, 2008.

(51) Int. Cl. A61K 31/7088; A61P 33/00; A61P 31/04; A61P 31/12; C12Q 1/68; A61P 31/00

(52) U.S. Cl. 514/44 R; 435/6.1

(57) ABSTRACT

Through ecological re-cycling, plants take up the nutrients from the cadavers. But it is not understood whether the plant roots can take the nutrients and the genes of the dead plants and cadavers, to develop into a transgenic plants.

Biotechnology has successfully developed transgenic plants and animals. Recently, gene sequencing studies, have shown that animals carry foreign genes making them as natural transgenic animals. Reason for the presence of transgenes not investigated. Further studies will enable us to learn more about the existence of transgenic flora and fauna and the role of gene re-cyling.

Corroborating the Nature's hidden biological phenomena, like ethnicity, cyclic occurrence of diseases, re-appearance of extinct animals, Biblical citations etc., with modern literature on transgenic plants/animals, human genomic studies. gene sequencing, etc. it is hypothesized that genes of flora and fauna are recycled from generation to generation through immortal DNA. It will change the present concept of organism's mortality to immortality.

Current literature on the cutting edge Biotechnology experimental findings, genetic engineering, gene sequencing and gene resequencing studies have enabled to hypothesize, the theory of “Gene re-cycling”, that genes of every flora and fauna are recycled from generation to generation through the transgenic plants, humans, animals, birds or fishes.

Gene re-cycling theory will reveal the scientific truth behind the natural biological phenomena occurring in our planet.

As per the natural phenomenon occurring and the modern literature cited below, plant's roots can attract the genes from the humus of other organisms in the soil/water which will then become an integral part of the plant chromosome.

Such foreign genes from other species of organisms, after integrating it into the plant chromosome, will remain inactive and enter into a latent period. Genes, after entering into a homologous host and under appropriate environmental conditions will become active again and multiply with the gene expression of the original homologous host.

### SUMMARY

This is a new theory invented/hypothesized. It has great utility for the following purposes:
1. Knowledge of disease outbreak/infection caused by the genes of pathogenic organisms transported from soil and water via transgenic plants/fishes to humans and animals, can help to take appropriate disease control measures,
2. Revealing the root cause of biosphere ethnicity due to genes in the food from Transgenic plants and animals,
3. Understanding the effect of expression of genes in the food on physiological and genetic changes in humans, animals and plants.

### Revised Experimental Proof

### Introduction:

In the earlier Provisional Application dated Sep. 30, 2008, an experiment was planned to conduct the Gene sequencing and re-sequencing of human genome to prove the Gene Recycling and development of ethnicity. On my preliminary enquiry with gene sequencing scientists, it was found out that human genome has more than 1 million base pairs and the cost of sequencing and re-sequencing of human genome will be very high, since sequencing each base pair will be around ¢5 per base pair.

Hence it was decided to plan the experiment of gene re-cycling, with beef cattle (meat) having less number of genome base pair (roughly 22,000 genes). Sequencing of cow's Genome has been studied and is available in the Genome bank.

Current literature: In the AARP bulletin today (http://bulletin_aarp.org/your/health/policy/articles/scientists _unravel_genome_of_the_cow.ht.8/82009 it was reported that “Of cow's 22,000 genes versions of at least 14,000 have counterparts in other mammals. Cows appear to have 1000 genes that they share with dogs and rodents (Brown, and Lewin 2009)”. It shows that 1000 latent genes of heterologus species (dogs and rodents) that are not found in people, might have entered the cow's genome through gene recycling, developing the animal into a transgenic cow. This transgenic cow, hidden in her roughly 22000 genes are hints of how natural selection/gene re-cycling, sculpted a bovine, with full gene expression over the past 60 million years (Brown, D and Lewin, H. A. 2009). It shows that 1000 latent genes of heterologus species (dogs and rodents) that are not found in people, might have entered the cow's genome through gene re-cycling, developing the animal into a transgenic cow. This transgenic cow, hidden in her roughly 22000 genes are hints of how natural selection/gene re-cycling, sculpted a bovine, with full gene expression over the past 60 million years (Brown and Lewin, 2009)”. This scientific report indicate that natural biological Gene re-cycling results in the development of transgenic organism, creating new species of a unicellular/multicellular organism (extinct animals and birds) through Gene re-cycling take place with long periods of time, depending upon the biology of the organism.

The above scientific literature fully proves/support the theory of Gene Re-cycling, as transgenic animals are being developed naturally in the development of new life on Earth. When the genes, recycle through inter species and intra species, it makes the genes immortal with or without mutation. As time goes many such articles may be published proving gene re-cycling is not a Hypothesis, but a proven naturally existing scientific truth. This made me to submit a Non-provisional Patent Application.

Meanwhile I have also planned an experiment, which may not be necessary as per the scientific proof given in the paragraph [0008 and 0009] given above to produce transgenic cereal plant by planting the cereal seeds in soil buried with beef meat (putrefied). After conducting the squencing and re-sequencing studies of the beef cattle genes and that of cereals, the transgenic genotype of the cereal will be decided.

After confirming the transgenic nature of the cereal it will be fed to the mice/rabbits and observe those experimental animal for the transgenes from the beef cattle. The result will be published in scientific journal/scientific conference.

In the light of the above a Non-Provisional Application on “Gene re-cycling” as given below, has been submitted as a continuation of Utility Provisional application No. 61/194,637 Dt. Sep. 30, 2008 for Patent registration.

Plants take up the nutrients from the dead and putrefied flora (humus) & fauna, recycle it for the nutrition and growth of humans, plants and animals, and thus maintain ecological re-cycling system. Similarly the herbivores and carnivores animals also play their own role in the ecological recycling process. However more scientific data is required to know, whether the plants can take not only the nutrients but also the genes/viruses/bacteria from the infected materials, dead animals, humans, birds or plants in the soil & water and recycle those genes, generation after generation making the biosphere as immortal. Hence it is hypothesized that genes of every flora and fauna are recycled from generation to generation through the plants, humans, animals, birds and fishes (living in land, sky and water).

When the “Gene Recycling” hypothesis is proved the concept of mortality of living beings will be changed to immortality. This is a postmodern century of interrogation. “Gene Recycling” hypothesis can provide full or partial answer for many of the Mother nature's biological phenomenon we now see in the world.

Based on the modern scientific literature and technology, the theory of “Gene Recycling”, can provide a strong support to the natural unknown biological phenomena occurring in our planet. It is in the hidden plan of the Creator for making the biosphere/life on this planet as immortal. As per the natural phenomenon occurring and the modern literature cited below, plant's roots can attract the genes from the humus of other organisms in the soil/water which will then become an integral part of the plant chromosome. Such foreign genes from other species of organisms, after integrating it into the plant chromosome will remain inactive and enter into a latent period. Genes, after entering into a homologous host or under appropriate environmental conditions will become active again and multiply with the gene expression of the original host. The scientific literature given below provide s a strong support to the “Gene Recycling” hypothesis.

### Natural Biological Phenomena:

1. Prevalence of Ethnic Population and Evolutionary Conservatism.

All organisms on Earth appear to be unrelated in the evolutionary process of different species of plants, birds, animals, mammals and humans. But a detailed study of the molecular biology of cells, has shown that basic molecular structure of all living organisms are conserved to a great extent and all have the same chemical structure such as amino acids, sugars, fatty acids and nucleotides. Besides the above, other resembling factors are that the genetic information of all multi cellular organisms, stored in the genome, is carried by DNA and is transported, from generation to generations. The hereditary information is stored in all the cells in the DNA as chemical code, arranged in linear fashion (Koonin, 2002).

We can also notice the family resemblances among genes coding for proteins. Genes and portions of genes get duplicated to develop into new diverse genes through mutation of the parent genes. Usually one cell expresses only a fraction of its genes. Changes in the environment due to signals from other cells can influence the pattern of genes they express. The gene expression by different sets of genes produces different types of cells in a multicellular organism. Thus a small group of genes may multiply to develop into higher plant or animal species with large number of genes. A broad phylogenic picture provides a rough idea of the relation ship between the species on our planet Earth (Li, W-H and Graur, 1991).
1. (a) “Gene Recycling” Hypothesis Claim:

If the roots of the plants are taking organic and Inorganic nutrients along with the genes from the human and animal cadavers, then the plant and its parts or fruits will also contain these human, animal genes, which will remain as tent genes. The plant/its parts or fruits containing such foreign (human/animal) latent genes, when consumed by other people in the locality or town, the genes may enter their gastro intestinal system and reproductive system (gonads). It may then remain in the system as latent genes. These latent foreign genes which have entered into the gonads may pass it on to their progeny and become active and provide gene expression (dominant genes) in the next generation of people in the locality/town. This will result in ethnicity (common characters related to place of origin, culture, speech, food habits etc.) in those local people.
1. (b) Supporting literature:
1. (b) 1. Transgenic plants:

Genetic engineering studies have revealed that a mutant gene can be inserted into the chromosome of a plant cell and it becomes a part of the plant genome. In the laboratory experiment it has been shown that, when the tobacco leaf discs incubated in a selection media with genetically engineered Agrobacteria, the injured cells at the edge of the plant leaf disc, released substances that attract the Agrobacteria which carry a recombinant plasmid with a selectable marker and desired transgene, inject its bacterial DNA into the plant cells. The plant cells which take up the appropriate bacterial DNA, survive and proliferate to express the selectable marker gene, and develop into a callus. When these calluses are placed in special medium, it will induce the production of shoots. These then develop into adult plants carrying transgenes (Davey et al., 1989 & Walden and Schell, 1990).
1. (b) 1.1. Transgenic Plant Experiment Supports the Hypothesis:

Similar to the experiment stated above if an injured cell from the plant leaf is attracting the DNA from the bacteria; as per the hypothesis, under natural environmental conditions, the injured cells of plant roots can also attract/absorb/take up the gene(s) along with the nutrients from the dead and putrefying flora (humus) and fauna in the soil/water. When such a foreign gene from other species is recognized by a plant cell, it will efficiently transfer a DNA strand of human/animal/bacterial/viral gene to the plant cell. It then becomes the part of plant chromosome, survive to grow into a transgenic plant carrying such a foreign gene.
1 (b) 1. 2. Possibility 1:

The transgenic plants (or its product) carrying foreign gene when consumed by species homologous to the gene, it can develop to express the phenotypic and genotypic characters of the gene. In other cases it may remain as latent gene and express it only when it enters into a host homologous to the gene.
1. (b) 1. 3. Possibility 2:

The transgenic plants (or its product) carrying foreign gene when consumed by species heterologous to the gene, it may remain as a latent gene and will pass it on to its progeny and continue to remain as latent gene in the progeny.
1. (b) 2. Transgenic animals:

In the case of mouse, foreign DNA can be randomly integrated into the genome. When fragments of DNA are introduced into cells, they are rapidly joined end to end by intra cellular enzymes and form long tandem arrays. These are then randomly integrated into a chromosome. When original gene(s) of an animal, is thus altered permanently, such an animal is known as transgenic animal. The genes which are responsible for altering are called transgenes (Boyd and Samid, 1993 & Palmiter and Brinster, 1986).
1. (b) 3. Role of Gene Regulatory Proteins:

Genome (whole set of genetic information belonging to a cell/organism carried by DNA) in its DNA sequenquence carries the information to express many thousands of different proteins and RNA molecules. Usually cells express only a fraction of its genes. The gene expression by different sets of genes produces different types of cells in a multicellular organism.

Changes in the environment such as signals from other cells can influence the pattern of genes they express. Even though the expression of a gene is generally regulated, the initiation of RNA transcription will be an important control point for most genes. The transcription of the gene in the DNA is controlled by the regulatory region at which it starts. These regulatory regions function like tiny microprocessors. Thus these regions act like switches by responding to the various signals, which they interpret and integrate the neighboring gene to switch on or off. Bacterial Genetic study conducted in 1950 gave the first evidence on the existence of “gene regulatory proteins”. These gene regulatory proteins, turns specific sets of genes on or off, the production of proteins. These are called “repressors” as they shut off the production of proteins. These repressors can be (1) viral encoded gene regulatory protein or (2) bacterial gene regulatory protein.
1. (b) 3. 1. Viral Encoded Gene Regulatory Protein

The bacteriophage lambda repressor, is a gene regulatory protein encoded by a bacterial virus. It shuts off the viral genes that code for the protein components of new virus particles. Thus it enables the viral genome to remain as silent occupier in the viral chromosome and multiplies along with the bacterial growth. Lambda repressor is one of such regulatory proteins which are encoded by bacteriophage lambda. This lambda repressor shuts off the viral genes that code for the protein components of new virus particles, and there by makes the virus genome inactive in the bacterial chromosome. But the mutant bacteriophage lambda cannot shut off the specific set of genes. With the help of DNA sequencing and DNA foot print studies it has been found that these regulatory proteins are bound to specific DNA sequences close to the genes which they regulate (Gilbert and Muller-Hill, 1967; Jacob and Monod, 1961; Kaiser, 1957 & Ptashne, 1967).
1 (b) 3. 2. Bacterial Gene Regulatory Protein:

The lac repressor is the first bacterial gene regulatory protein to be recognized. These generally respond when there is a nutritional requirement and they respond by shutting off genes encoding specific sets of metabolic enzymes when they are not necessary. Thus when sugar is absent in the medium, lac repressor turns off the production of the proteins responsible for lactose metabolism, (Jacob and Monod, 1961 & Gilbert and Muller-Hill, 1967).
1. (b) 4. Growth Factors Regulate the Protein Synthesis:

The growth factors regulate the protein synthesis and stimulate the cell/proliferation. Most growth factors also stimulate cell growth. (Loughlin and Fallon, 1992 and Purves, 1988).
1. (b) 5. Cells Enter Into Non Growing State—“G Zero” in the Absence of Growth Factors:

Mammalian cells stopped growth between mitosis and S phase, when they are deprived of serum which contains highly specific proteins called Growth Factors. In the absence of serum these, cells will delay division by entering a specialized non growing state called “G zero” (Pardee, 1974 & 1989).
1. (b) 6. Cell Senescence:

Cells from mammalian tissue cultures can be propagated only for a certain number of cell divisions (50 divisions for human cell cultures), after wards they cease to divide and die. This process is called cell senescence. But some cells derived from rodent tissue cultures escape senescence and divide indefinitely to produce cell lines (Todaro and Green, 1963).
1. (c) Hypothesis Linked to the Above Cited Literature:

Similar to the reference cited above the mammalian gene(s) taken up by the roots of the plants can remain as silent occupier, in the plant/heterogenous species, as the gene regulatory proteins switch off the foreign genes, or the cells may cease to divide due to cell senescence. It will only multiply and provide the original specific gene expression, . with phenotypic and genotypic characters, if it enters a homologous host and under suitable internal or external environment.

When the root of the plant takes up the (dominant) genes of other species from the soil, these foreign (to the plant) genes will lie in a latent state as lambda bacteriophage is integrated into E. Coli. These genes foreign to the plant may then enter into other species (humans/animals) through the consumption of the plant products, containing genes of other species and will be passed on to a different species (humans/animals/birds). The gene regulatory proteins can switch on or off specific sets of genes, depending upon the homologous host cell in which the gene is integrated. Parmley et al. ( 2007) recently reported that a secondary function of some pieces of DNA has the capacity to hold back the function of production of the protein, by keeping a check and slowing the process of associated genes, becoming ideal protein machines.

Experiments proposed: Experiment 1. 1. Study to find out whether the plant roots take up the human genes from the soil and get integrated into plant chromosomes.

### Step 1. Objective:

To find out whether the plant roots will take up the human genes through its root and get it integrated into the plant chromosome.

Method: Conduct a DNA test of the person who is willing to participate in this study, using the commercially available ISO approved test kit. “The test will have the 16 DNA genetic markers including the 13 markers used by the FBI and other law enforcement agencies for human DNA testing. The 13 markers are referred to as the Combined DNA Index System (CODIS). All of these 13 markers are used only in the determination of human identity” (GTL, 2008). Conduct the experiment planting the seeds of paddy, wheat or carrot, in fresh soil containing only chemical fertilizer and organic nutrients from placenta, blood and or urine sourced from the above single person with known DNA profile.

### Step 2.

At the fruit bearing age of the plant, the parts of the plant like leaves, stem, fruits etc. will be collected and send for gene testing/mapping, to the Plant Genome Mapping Laboratory (PGML), University of Georgia. If the results show that the human genome is integrated into the plant chromosome and present as latent human gene in the seeds of paddy, wheat or carrot, the following experiment will be conducted.

### Experiment 1. 2. 1.

Study to find out whether the latent human transgenes in the plants are transmitted to animals through consumption of transgenic plant products (underground stem/fruits).

### Objective:

To find out whether the underground stem/fruit of the transgenic plants carrying the human genes in the plant chromosome will be present as latent gene in the blood/gonads of mice/rabbits, when the transgenic plant product (rice, wheat carrot) are consumed by mice/rabbits.

### Method: Experiment 1. 2. 1. 1. (Transgenic Wheat Plant):

Take one group of six (3 male and 3 female) Swiss albino mice of 3 weeks old. Keep them in separate cage with tag No. Collect the blood and conduct DNA test for base line data. Feed each mouse with wheat (harvested from the experiment 1.1, containing the human genes as integrated part of the plant chromosome) and water for 3 months. Collect duplicate sample of blood from each mouse at month interval and conduct DNA test to find out the presence of human latent gene.

Experiment 1. 2. 1. 2. (Transgenic Rice plant):

Take second group of six (3 male & 3 female) white mice of 3 weeks old. Keep them in separate cage with tag No. Collect the blood and conduct DNA test for base line data. Feed each mouse with rice (harvested from the experiment 1.1—containing the human genes as integrated part of the plant chromosome) and water for 3 months. Collect duplicate sample of blood from each mouse at month interval and conduct DNA test to find out the presence of human latent gene.

Experiment 1. 2. 1. 3. (Transgenic carrot):

Provide one group of six (3 male & 3 female) rabbits of one month old, each with carrot (harvested from the experiment 1.1—containing the human genes as integrated part of the plant chromosome) for 3 months. Keep them in separate cage with tag No. Collect the blood and conduct DNA test for base line data. Feed each mouse with carrot (harvested from the experiment 1.1) and water for 3 months. Collect duplicate sample of blood from each rabbit at month interval and conduct DNA test to find out the presence of human latent gene.

### Experiment 1. 3.

Study to find out whether the human latent genes noticed in the blood of transgenic mice/rabbits will be transferred to their offspring.

### Experiment 1. 3. 1.

At the end of the 3 months experiments, the animals whose blood showed positive for human latent gene will be grouped (separate for each food) together to breed and produce offspring. After weaning, test the blood of the offspring for human latent gene. Then euthanize the parent animals to harvest the gonads (testes and ovary) and send them for DNA testing, to confirm human latent genes in the gonads.

### CONCLUSION

Finding human latent gene in the mice/rabbits and their progeny will be a proof of the “Gene Re-cycling”.

II. Natural phenomena:
Cyclic development of viral diseases.

Present scientific view is that the pathogenic bacteria/viruses exist in animals or birds as they become carriers of bacteria/viruses (host—parasite balance) without producing disease. Under adverse environmental conditions the disease outbreak occurs, due to the break down of immunity in those carrier animals/birds causing the cyclic development of disease outbreaks. Friedman (1992) reported that viral chromosomes can integrate into host chromosomes. The viruses which are integrated into the plant chromosome need not multiply immediately, but goes into a latent state and remain as inactive in the plant cells as provirus. It will come active only under suitable conditions sometimes after passing through several hosts/generations.

Hypothesis: The viruses/bacteria in the dead bodies of the humans/animals or the human/animal excreta, will be taken up by the roots of the plants grown in the soil in that area, get integrated into the plant chromosomes. The cyclic outbreak of certain viral diseases can then be attributed to the release of the viruses integrated into the plant chromosome, when such plants or its part/fruit are consumed by susceptible (immune compromised) humans or animals, resulting in an infection in the people/animals leading to fresh outbreak of the viral/bacterial infection in that area. The delay in the outbreak may be due to the time the virus/bacteria takes to travel from the roots to fruit/plant parts and the chance occurrence of a susceptible person/animal, eating the affected plant part. It is also important to note that certain plants like coconut trees/jack fruit trees etc. will take 5-8 years to bear fruits.

### II. Supporting Literature:

1. Viral Chromosomes Integrate Into Host Chromosomes:

The viral chromosomes after entering a host cell, necessarily need not multiply to produce more viral progeny. Because the genomes of these viruses enter into a latent state and become inactive in the host cell without producing any progeny. This viral latency was found out when uninfected bacteria, on exposure to UV light was able to induce the production of progeny of bacteriophages. Subsequently it was shown that these uninfected bacteria (lysogenic bacteria) carry in their chromosome, a complete viral (bacteriophage) chromosome lying in a dormant state. These types of host integrated viral chromosomes are called “provirus”. Bacteriophages which can integrate their DNA into bacterial chromosomes are called as temperate bacteriophages. Bacteriophage lambda is a temperate bacteriophage and when it infects a suitable E. coli host cell, it can grow either by lytic or lysogenic pathway. In the lytic path way lambda multiplies to several progeny particles and they will be released when the E. coli is lysed. In the lysogenic pathway, the integrated viral DNA replicates along with the host chromosome and produce new integrated viral DNA copies, resulting in the development of a lysogenic bacterium. This lysogenic cell multiplies in the normal way. But when it is exposed to any environmental insult/changes like exposure to UV light, or ionizing radiation, the cell becomes fragile/weakened leading to the release of the integrated provirus from the cell chromosome. Then it will lead a normal cycle of viral multiplication. It shows that the integrated provirus will not perish along with its damaged host cell, but can escape to infect other E. coli Campbell (1993).

Hypothesis: As per the literature cited above the mammalian/human gene(s) taken by the roots of the plants can integrate itself into plant chromosome and will remain as an integrated gene. The integrated foreign gene will be released from the plant chromosome, when the plant products are consumed by humans/animals etc. or cause any physical or chemical injury to the plant cell due to any environmental changes like extreme heat or cold or change in the nutritional status of the plant. Then the gene released from the plant chromosome will then follow the lytic or lysogenic path way and will lead to the escape of the released gene to other species. When the gene enters a heterologous unrelated species of organism, the gene regulatory proteins will switch off the foreign genes entered into unrelated host. If the gene released from the plant cell entered into a new suitable homologous species, related to the gene's original host, the gene regulatory proteins will be switched on.

To explain it further, the human gene integrated into the plant cell chromosome, when released by the consumption of humans, the gene(s) will enter a new cell in the human body, it will be expressed providing the phenotypic and genotypic characters of the gene remained in the plant chromosome as integrated gene.

III. Natural phenomena: Transmission of Prions (PrP Gene) Through Food and Hereditary:

Feeding “Renderers meat” with sheep offal, contaminated with Scrapie (PrPsc) produce BSE infection/mad cow disease in beef cattle. Consumption of beef infected with BSE/TSE prion (aberrant protein) will produce vCJD infection in humans. The causative agent of BSE/TSE is believed to be a small protein infectious particle (prion) which resist inactivation by the methods capable of destroying nucleic acids. The recent discovery that only a protein can cause and transmit the disease is shocking to the molecular biologists and virologists.

Hypothesis: As prions are transmitted through food and hereditary, the genes integrated into the plant chromosome when consumed by the animal or humans, will also produce gene expression in the animals/humans, if the gene is homologous or continue to remain as latent gene as in the case of prions (production of a slow latent infection).

### III. 1. Supporting Literature:

Feeding “Renderers meat” with sheep offal, contaminated with ovine prion protein (PrP) gene in scrapie (Shiquan, et al. (2002) produce BSE infection/mad cow disease in beef cattle. Consumption of beef infected with BSE/TSE prion (aberrant protein) will produce vCJD infection in humans. Humans can be infected by Prions either as acquired through diet and surgical procedures or through “apparent hereditary Mendelian transmission where it is an autosomal and dominant trait” (Alan Cann, 2005). Prions are responsible for a slow developing disease due to the latent infection. It can be compared to the latent gene/host integrated viral chromosomes or provirus/bacteriophage lambda (Campbell, 1993).

### IV. Natural Phenomena/Mythology:

Re-incarnation myth becomes a scientific reality due to modern scientific biotechnology success in cloning, and Transgenesis.

In India there are so many stories of re-incarnation/re-birth of a person in another part of India or world. Re-cycling of genes is probable explanation for the myth of re-incarnation. It is possible to explain hypothetically that the dominant genes of “X” person's cadaver integrated into the soil, when taken up by the root of the plant, will remain as latent genes in the plant part/fruit. If the plant part is then taken up by another person and entered into the gonad of another person, the phenotypic and genotypic character of the dominant gene of the “X” person will be expressed in the progeny as rebirth/reincarnation of the “X” person.

Re-incarnation story give strong support to the Gene Recycling Hypothesis.

Gene re-cycling hypothesis can be the scientific/nature's explanation for the Hindu mythological stories on people with animal face/head and human body or female body with fish tail (Mermaid) manifested as nature's freak, due to incomplete expression of human gene(s), integrated into animal chromosome (transgenic animal) or vice versa. Traditionally many people attribute such nature's freak is endowed with God's power Cherubim is a typical example for transgenic person with incomplete expression of genes from other species like animals & birds. It is also possible that the human gene is carried by another animal as shown in the movie of the dog “Fluke”.

### V. Natural Phenomena:

Reappearance of the Extinct Animals and Birds:

Cell theory of 19^{th} century stated that all living organisms possess cells and the new cells are developed only from the existing cells. Thus all Eukaryotic or Prokaryotic unicellular or multi cellular organisms are developed through the evolution from an original population of primitive “cells”.

Hypothesis: New cells will be developed by recycling the genes of the previous generation of living organisms existed earlier on earth. The well-known saying “Life comes after death” should be a scientific fact. The animals and birds once lived on earth after extinction may come back again to life through the recycling of the genes deposited in the humus and remained as integral part of the soil. The plants grown on such soil with the genes of the extinct animals and birds, may take up the genes through its roots and can remain in the leaves and fruits. When animals/birds of other species eat the leaves/fruits, the genes of extinct animals/birds, may enter the body of the new host and by entering the ova/sperm may re produce the life of the extinct species. Recently it has been reported that Black footed Ferret which was extinct has been found recently.

### VI. Biblical References: Genes in the Trees, Stones and Bones:

### VI. A. Genes in the Tree—Tree of Life: Garden of Eden:Bible (1973). Genesis Ch. 2, Verse 8 and 9.

“8. And the Lord God planted a garden toward the east, in Eden; and there He placed the man whom he had formed.

“9. And out of the ground the Lord God caused to grow every tree that is pleasing to the sight and good for food; the tree of life also in the midst of the garden, and the tree of the knowledge of good and evil”.

### VI. B. Forbidden Fruit: Does the Fruit From the Tree in the Garden of Eden Contained Gene to Know Good and Evil?

Bible (1973). Genesis Ch. 3. verse 2-5.
“2 And the woman said to the serpent, “From the fruit of the trees of the garden, we may eat;

“3. but from the fruit of the tree which is in the middle of the garden, God has said, ‘You shall not eat from it or touch it lest you die!

“4. And the serpent said to the woman, You surely shall not die!

“5. For God knows that in the day you eat from it your eyes, will be opened and “you will be like God, knowing good and evil”.

VI. C. Gene expression—Tree of Life: Disobedience and Fall of Man and Woman from God's Grace: Bible, 1973: Genesis Ch. 3, verse 22-24.

“22 Then the lord God said, “Behold, the man has become like one of Us, knowing good and evil; and now, lest he stretch out his hand, and take also from the tree of life, and eat and live forever”—

“23 therefore the Lord God sent him out from the Garden of Eden, to cultivate the ground from which he was taken.

“24 So He drove the man out; and at the east of the garden of Eden He stationed the cherubim and the flaming sword which turned every direction, to guard the way to the tree of life”.

Hypothesis: The above Biblical passage gives us the insight that God intended that man and woman to lead an eternal life by eating the fruit of “the Tree of Life”. But since man and woman disobeyed God and sinned, He threw them out of the Garden so that they will not have eternal life, simply by eating the fruit. Instead they (we) have to toil in the soil and go back into the soil, so that our gene will integrate with the soil and will regenerate again into humans with the gene integrated in the trees/plants/fruits rooted in the same soil. Thus each tree by taking the human genes from the soil with their roots, become the symbolic of the tree of life of the Garden of Eden.

Thus through the recycling of genes through the plants/tree of life, humans have eternal life and will have several opportunity to become self purified to receive the

Lord Jesus when He comes 2^{nd} time. It can be presumed that each civilization on this earth is a re-incarnation of the previous civilization. If each person on this earth is a new entry into this planet, God must have created several billions or trillions of people, but through recycling of genes most probably, we are all born repeatedly on this earth (re-incarnation/rebirth?).

### VI. D. God Can Raise Up Children From Stones:

Bible (1973). New Testament; Matthew Ch. 3, verse. 9 “and do not suppose that you can say to yourselves, We have Abraham for our father; “for I say to you, that God is able from these stones to raise up children to Abraham”. We cannot simply ignore this as a statement by John the Baptist out of his anger or frustration. If we read it in between the lines, then we can understand or presume, that the soil and the stones may contain the genes of the human beings, hence John the Baptist knowingly or unknowingly said that God can raise the people from the stones/soil.

Hypothesis: If we understand that every flora and fauna seen on the earth are mortal and become part of the earth after their life span, then we should also know that new life on earth starts not only by recycling the nutrients but also by genes of the dead flora and fauna.

The well known fact: “LIFE COMES AFTER DEATH”, will be supportive of the hypothesis that genes of every flora and fauna are recycled from generation to generation.

VI. E. Ezekiel's Prophesy of Life from Dry Bones:
Bible (1973). Ezekiel—Book of Ezekiel: Ch. 37. verse 1-14,

“The hand of the lord was upon me, and He brought me out by the Spirit of the Lord and set me down in the middle of the valley; and it was full of bones.

“2 And He caused me to pass among them round about, and behold, there were very many on the surface of the valley; and lo, they were very dry.

“3 And He said to me, “Son of man, can these bones live?” And I answered, “0 Lord God, Thou knowest.”

“4 Again He said to me “Prophecy over these bones and say to them, O dry bones, hear the word of the Lord.”

“5 Thus says the Lord God to these bones, Behold I will cause breath (spirit) to enter you that you may come to life.

“6 And I will put sinews on you, make flesh grow back on you, cover you with skin, and put breath in you that you may come alive; and you will know that I am the Lord.”

“7 So prophesied as I was commanded and as I prophesied, there was a noise, and behold a rattling; and the bones came together, bone to its bone.

“8 And I looked and behold, sinews were on them and flesh grew, and skin covered then: but there was no breath in them.

“9 Then He said to me “Prophecy to the breath, prophecy son of man, and say to the breath, “Thus says the Lord God, “Come from the four winds, O breath and breathe on these slain, that they come to life.”

“10 So I prophesied as He commanded me, and the breath came into them, and they came to life, and stood on their feet, an exceedingly great army.

“11 Then He said to me “Son of man, these bones are the whole house of Israel; behold they say, “Our bones are dried up and our hope has perished. We are completely cut off.

“12 Therefore prophecy and say to them “Thus says the Lord God, “Behold I will open your graves and cause you to come up out of your graves, My people; and I will bring you unto the land of Israel.

“13 “ Then you will know that I am the Lord, when I have opened your graves and caused you to come up out of your graves, My people”.

“14 “And I will put My Spirit within you and you will come to life, and I will place you on your own land. Then you will know that I the Lord have spoken and done it, “declares the Lord.”

### SUMMARY

By applying modern science and technology knowledge, it is hypothesized that “Genes of every flora and fauna are recycled from generation to generation through the plants, humans, birds and animals living in land and water”. Gene Recycling plays the most crucial role in the continued propagation of flora and fauna on our planet. Through gene recycling, biosphere is made to immortal existence. This is a postmodern century of interrogation. Gene Recycling Hypothesis can provide full or partial answer for many of the nature's phenomenon we now see in the world.

### REFERENCES

Alberts, B.; Bray, D.; Lewis, J.; Raft M.; Roberts, K, and Watson, J. D. (1994). Molecular Biology of THE CELL. 3rd Ed. Pub. By Garland Publishing Inc. 717 Fifth Avenue New York, N.Y. 10022.

Alberts, B.; Alexander, J.; Julian, L.; Martin, R.; Keith, R. and Peter,W. (2002). Molecular Biology of THE CELL 4^{th} Ed. Pub. By Garland Science, a member of the Taylor & Francis Group, 29 West 35^{th} Street, New York, N.Y. 1001-2299.

Bible, (1973). Genesis-In: Chapter 2. verse 8 and 9. p. 3; New American Standard Bible. Reference Edition; Foundation Press Publications Box 277, La Habra Calif. 90631.

Bible, (1973). Genesis-In: Chapter 3. verse 22-24. p. 5. New American Standard Bible. Reference Edition; Foundation Press Publications Box 277, La Habra Calif. 90631.

Bible, (1973). Ezekiel—Book of Ezekiel.In: Chapter 37. verse 1-14, p. 1213. New American Standard Bible. Reference Edition; Foundation Press Publications Box 277, La Habra Calif. 90631.

Bible, (1973). New Testament. Matthew. In: Chapter 3. verse. 9. p. 3 New American Standard Bible. Reference Edition; Foundation Press Publications Box 277, La Habra Calif. 90631.

Boyd, A. L. and Samid, D. (1993). Review: Molecular biology of transgenic animals. J. Anim. Sci. 71(S3): 1-9.

Brown, D. and *Levine, H. A. (2009) “Scientists Unravel Genome of the Cow” Washington Post Staff Writer: Friday, Apr. 24, 2009. And in the AARP Bulletin: (http://bulletin aarp.org/yourhealth/policy/articles/scientists _unravel_genome_of_the_cow.ht.8/8200); *Head of the Institute of Genomic Biology, Uni. Illinois, Urbana-Champaign.

Campbell, A. M. (1993). “Thirty years ago in genetics: prophage insertion into bacterial chromosomes”. Genetics 133 (3): 433-438.

Cann, A. J. (2005). In: Chapter 8. Principles of Molecular Virology (Standard. Edition) Fourth Edition (Kindle Edition) Academic Press. (cited In: http://www.microbiologybytes.com/virology/prions.html

Davey, M. R.; Rech, E. L and Mulligan, B. J. (1989). Direct DNA tranfer to plant cells. Plant Mol. Biol., 133: 273-285.

Friedman, D. I. (1992). Interaction between bacteriphage lambda and Escherichia coli host Curr. Opin. Genet. Dev., 2: 727-738.

Gilbert, W, and Muller-Hill, B. (1967). The lac operator is DNA. Proc. Natl. Acad. Sci., USA, 58: 2415-2421.

GTL-(GeneticTestingLaboratoriesInc.)2008. https://www.gtldna.net/whysocheap.html

Jacob, F. and Monod, J. (1961).Genetic regulatory mechanisms in the synthesis of proteins. J. Mol. Biol., 3:318-356.

Kaiser, A. D. (1957). Mutations in a temperate bacteriophage affecting its ability to lysogenize, E. coli. Virology, 3: 42-61.

Koonin, E. (2002). Cells and Genomes. (cited In: p. 3 Alberts et al., 2002).

Li W-H. and Graur, D. (1991). “Sequence studies reveal resemblances between several families with Homologous genes”: Fundamentals of Molecular Evolution Sunderland, M. A. Sinauer Associates. (cited In: p. 281 “Molecular Biology of THE CELL” 3rd Ed. Alberts et al., 1994).

Loughlin, J. H. and Fallon,. J. H. (1992). “Neurotrophic Growth Factors”. San Diego, Calif. Academic Press. “Cell Growth and cell division can be independently regulated”. (Cited in p. 894 “Molecular Biology of THE CELL” 3rd ed. Alberts et al., 1994).

Palmiter, R. D. and Brinster, R. L. (1986). Germ line transformation of mice. Anim. Rev. Genet., 20: 465-499.

Pardee, A. B. (1974). A restriction point for control of normal animal cell proliferation. Proc. Natl. Acad. Sci. USA, 71: 1286-1290. Pardee, A. B. (1989). G1 Events and regulation of cell proliferation. Science, 246: 603-608.

Parmley, J. L. and Urrutia, A. O. (2007). In: “Putting breaks on evolution—Layered information holds back protein evolution” by Helen Pearson, newa@nature.com 5 Feb. 2007, doi:10. 1038/news 070205-3). By: Potrzebowski, J., Kaessmann, H. & Hurst, L. D. Plos. Biol., 5 e 14.

Ptashne, M. (1967). Specific binding of the λ phage repressor to λ DNA. Nature, 214: 232-234.

Purves, D. (1988). Body and Brain: A Trophic Theory of Neural Connections. Cambridge: Harvard University Press. “Cell Growth and cell division can be independently regulated”. (cited In: p. 894 “Molecular Biology of THE CELL 3rd Ed. Alberts et al., 1994).

Raff, R. A. and Kaufman, T. C. (1983). Embryos, Genes Evolution. New York: Macmillan, (cited In: Alberts et al p. 32,33 & 34. 1994).

Reed Wickner, (2008). “Prion Diseases” Microbiology Bytes, Internet updated Jan. 14, 2008.

Ruby, S. W. and Abelson, J. (1991). Pre mRNA splicing in yeast. Trends Genet., 7: 79-85.

Shiquan, W.; Noelle, E. C.; Janice, M. M.; Tracy, L. S.; Alma, M.; Diane, L. S.; Warren, C. F.; Gilbert, R. H.; Ronald, C. E.; Thomas, D. B.; Jonathan, E. B.; Jay, W. C.; William, D. T. & Michael, R. M. (2002). Polymorphic distribution of the ovine prion protein (PrP) gene in scrapie infected sheep flocks in which embryo transfer was used to circumvent the transmission of scrapie. J. Theriogenol. 67:p. 1865-1875. http://wwwtheriojournal.com/articlesgenol. 67:p. 18651875. http://wwwtheriojournal.com/article/S0093-691X(02)00673-8/abstract.

Todaro, G. I. and Green, H. (1963). Quantitative studies of the growth of mouse embryo cells in culture and their development into established cell lines. J. Cell Biol., 17:299-313.

Walden, R. and Schell, J. (1990). Techniques in plant molecular biology—progress and problems”. Eur. J. Biochem., 192: 563-576.

1. Reveal the root cause of ethnicity, due to foreign latent genes in the food and the immortality of the cells of flora and fauna through “Gene Re-cycling”.
(Explained in Specification No. 1. (1.1, 1.2, 1.3), 2 & 5 (5.1, .5.2 and 5.3).

2. “Gene Re-cycling” associate and help in the prevention of patho-physiological and genetic changes in humans, animals and plants, due to the expression of foreign genes in the food. It also associate and help in the prevention of the infection (bacterial, viral or parasitic) caused by the genes of pathogenic organisms in the soil and water, that are transferred via plant chromosome to humans. (Explained in Specification No. 3, 4 (4.1, 4.2, 4.3) & 5 (5.1, 5.2 and 5.3).
